SEQUENCE LISTING (10 sequences)

SEQ ID NO: 1 - DNA, 1781 bp, Actinobacillus succinogenes
  misc_feature: Zwf gene
gtcgacccgcctcggcagaaccggcgaatgggacaccgccgccggcgaactgttactggaagagctgggcggtaacgcgctggacgagcactatcaaccgctcacctataatcaacgggaaacctttgtcaatcctgatttcgtcatcactgccgatgcctcggtaaactggaaaaacgtctttcaatttaatttgccttagctattagattttttagcggaattcaggcattattaaccgactattttccagataaggattaggataaaaatgaaagcagaaaataattgtatcgtgattttcggcgcatcaggggatctgacgcaccgtaaattaattcccgcactctataatctttacaaaatcggacggttggaagaaaacttctccgtgctgggagtggcccgcacagaaatgacggatgacattttccgtgaaaaaatgcgaaccgccctgattacccaagaaaatgccgaaggcgaaacgctggataaattctgttctcatctgtattaccaggcggtaaacacctccgattcggcggattacgtaaagttactgcctcgtttggatgaattacacgacaaataccaaacctgcggcaatacgctttattatctatccactccgccgagcctgtacggcgttattccggaatgcctggcggctcacggcttaaatacggaagaattcggctggaaacggattatcgtggaaaaaccgttcggttacgatattaaaaccgccaaagcactggatattcagattcaccgtttcttcgaggaacaccagatttaccgtatcgaccattatttgggcaaagaaaccgtgcaaaatctgttggtgctgcgattctccaacggcctgttcgaaccgctttggaaccgtaacttcatcgattacgtagaaatcaccggcgcggaagagatcggcgtagaacaacggggcggctattatgacggttccggtgcaatgcgggatatgttccaaaaccacttattgcaagtattggcaatggttgccatggaaccgccggcgattattaacgccgattccatgcgtgacgaaaccgccaaagtgctctattgtctgcatccgttgaccacggaagatctcaaacacaatctggtattagggcaatacacggcctccaccgttgacgataaaccggtgaagggttatctggaggaagcgggcgtgccgtccgattccggcaccgaaacctacatggcgttgcgctgccaaatcgataactggcgctgggccggcgtgccgttttacgtgcgcaccggtaaacgcctgccgacccgggtgacggaaatcgtcattcatttcaaaaccacgccgcacccggtattcagccaaaatgcgccggataacaaattaatcatccgtatccaaccggacgaaggcatttccatgttcttcggtttgaaaaaaccgggagccggcttcgaggctaaagaagtatccatggatttccgttatgcggatatcagttcttccgctaatttattaaccgcctacgaacgtttactgcttgacgccatgaaaggcgacgccacattattcgcccgtaccgacgccgttcacgcctgctggaaattcgtgcagccgattttggattacaaggaaaaccaaggtcgcgtttacgaatatgaagccggcacccggggaccggtggaagcggataaacttatcgcccgtgaaggacgcgtatggcgcagaccttcgggctccatgaagaaaaaagcgtaaaccttacctggtatgaggatcc

SEQ ID NO: 2 - protein, 495 aa, Actinobacillus succinogenes
  MISC_FEATURE: Zwf protein
MKAENNCIVIFGASGDLTHRKLIPALYNLYKIGRLEENFSVLGVARTEMTDDIFREKMRTALITQENAEGETLDKFCSHLYYQAVNTSDSADYVKLLPRLDELHDKYQTCGNTLYYLSTPPSLYGVIPECLAAHGLNTEEFGWKRIIVEKPFGYDIKTAKALDIQIHRFFEEHQIYRIDHYLGKETVQNLLVLRFSNGLFEPLWNRNFIDYVEITGAEEIGVEQRGGYYDGSGAMRDMFQNHLLQVLAMVAMEPPAIINADSMRDETAKVLYCLHPLTTEDLKHNLVLGQYTASTVDDKPVKGYLEEAGVPSDSGTETYMALRCQIDNWRWAGVPFYVRTGKRLPTRVTEIVIHFKTTPHPVFSQNAPDNKLIIRIQPDEGISMFFGLKKPGAGFEAKEVSMDFRYADISSSANLLTAYERLLLDAMKGDATLFARTDAVHACWKFVQPILDYKENQGRVYEYEAGTRGPVEADKLIAREGRVWRRPSGSMKKKA

SEQ ID NO: 3 - DNA, 1558 bp, Actinobacillus succinogenes
  misc_feature: mdh gene
gaattcccgaagcgttcctgcgcgagtaacgctttaaaaacctgtaataggttatctgttttattgtcggtcataaatagaaatatatcagtttttaagtcgaaatattgcataaattctgcataaaaattcaaaattaatcaataaaaatttaagtttattgtgatttgagcgttttcgaaaaataaatgataaaaacttgttttagatcgtaaaaatagatgaatatttaattgagtttcattttttttcttcgtaaaatctacccagttcaagttattaatattatcgaggagtatctcatgaaagtaaccttattaggcgccagcggcggtatcggtcaacctctttcattgttgttaaaattacatcttccggcagaaagcgatttaagcttatacgatgttgcgccggtcacccccggtgtggcgaaagacatcagccatattccgacttcggttgaagtggaaggtttcggcggcgatgatccgtccgaggcattaaaaggggcggatatcgttttaatctgtgcgggtgtggcgcgtaagccgggtatgactcgtgcggatttgtttaatgttaacgccggtattatccagaatttagtggaaaaagttgcgcaagtttgcccgcaggcttgtgtttgcattatcactaatccggtgaactcgattattccgattgcggcggaagtgctgaaaaaagcgggcgtatacgataaacggaaattattcggtattactacgctggataccatccgttccgaaaaatttatcgtgcaagcgaaaaatattgaaatcaaccgtaacgatatttcagttatcggcggacattcaggtgtgacgattttacctttgttgtcacaaattccgcatgtggaatttaccgagcaggaattaaaagatttaactcaccgcatccaaaatgccggcaccgaagtggtagaagctaaagccggtgcgggttccgctacactttccatggcgtatgcggcaatgcgttttgtggtttccatggctcgcgcattaaacggcgaagtgattacggaatgcgcctatattgaaggcgacggtaaattcgcccgtttctttgcacaaccggttcgtttgggtaaaaacggcgtagaagaaattctgccgttaggtacattaagcgcatttgagcaacaagcgcttgaagcgatgttaccgaccttgcaaactgacattgataacggtgtgaaatttgttaccggcgaataattcaccaaaataatttaacaaaaccgattaaaggattaggtttttatgcaaacctaatcctttttgtttggtatcaatcagttaaaatccgccgtttgattaatgggaagctatataagattttagtattttatatagataaaaatagcgtggaagaaataaagtaatcctccacgcgtcttctcaaaatgtataaaaagtgcggtcaaaaattaatcgattttttattcatcctcgtttcttggcggtttaatcgccagtaaattacattttaacttactgataacatgttcggcggtgttgcctaacaggaattc

SEQ ID NO: 4 - protein, 312 aa, Actinobacillus succinogenes
  MISC_FEATURE: mdh protein
MKVTLLGASGGIGQPLSLLLKLHLPAESDLSLYDVAPVTPGVAKDISHIPTSVEVEGFGGDDPSEALKGADIVLICAGVARKPGMTRADLFNVNAGIIQNLVEKVAQVCPQACVCIITNPVNSIIPIAAEVLKKAGVYDKRKLFGITTLDTIRSEKFIVQAKNIEINRNDISVIGGHSGVTILPLLSQIPHVEFTEQELKDLTHRIQNAGTEVVEAKAGAGSATLSMAYAAMRFVVSMARALNGEVITECAYIEGDGKFARFFAQPVRLGKNGVEEILPLGTLSAFEQQALEAMLPTLQTDIDNGVKFVTGE

SEQ ID NO: 5 - DNA, 29 bp, Artificial sequences
  Artificial Zwf primer
aaaggatcctcataccaggtaaggtttac

SEQ ID NO: 6 - DNA, 29 bp, Artificial sequences
  Artificial Zwf primer
aaagtcgacccgcctcggcagaaccggcg

SEQ ID NO: 7 - DNA, 28 bp, Artificial sequences
  Artificial Mdh primer
ccgaattcccgaagcgttcctgcgcgag

SEQ ID NO: 8 - DNA, 27 bp, Artificial sequences
  Artificial Mdh primer
aagaattcctgttaggcaacaccgccg

SEQ ID NO: 9 - protein, 491 aa, Escherichia coli
  ref <308>: DATABASE ACCESSION NUMBER: UniProtKB/Swiss-Prot / P0AC53.1
  ref <309>: DATABASE ENTRY DATE: 2013-09-18
  ref <313>: RELEVANT RESIDUES IN SEQ ID NO: (1)..(491)
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 10 - protein, 312 aa, Escherichia coli
  ref <308>: DATABASE ACCESSION NUMBER: UniProtKB/Swiss-Prot / P61889.1
  ref <309>: DATABASE ENTRY DATE: 2013-05-29
  ref <313>: RELEVANT RESIDUES IN SEQ ID NO: (1)..(312)
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVAKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

(19) United States
(12) Patent Application Publication
(10) Pub. No.: US 2014/0093925 A1
(43) Pub. Date: Apr. 3, 2014

(54) RECOMBINANT MICROORGANISMS FOR PRODUCING ORGANIC ACIDS

(71) Applicant: THE MICHIGAN BIOTECHNOLOGY INSTITUTE, Lansing, MI (US)

(72) Inventors: Michael Guettler, Holt, MI (US); Robert Hanchar, Charlotte, MI (US); Susanne Kleff, Okemos, MI (US); Sachin Jadhav, Lansing, MI (US)

(73) Assignee: THE MICHIGAN BIOTECHNOLOGY INSTITUTE, Lansing, MI (US)

(21) Appl. No.: 14/044,505

(22) Filed: Oct. 2, 2013

Related U.S. Application Data
(60) Provisional application No. 61/708,998, filed on Oct. 2, 2012.

(51) Int. Cl. C12P 7/52; C12P 7/46

(52) U.S. Cl. CPC C12P 7/52; C12P 7/46
USPC 435/141; 435/252.3; 435/136; 435/145

(57) ABSTRACT

Recombinant microorganisms that co express enzymatic glucose-6-phosphate dehydrogenase and malate dehydrogenase are generated to produce organic acids.

### STATEMENT REGARDING U.S. GOVERNMENT SUPPORT

Aspects of this invention were made with support of the United States government under the grant DE-FC36-02GO12001.

### CROSS-REFERENCE TO RELATED APPLICATIONS AND INCORPORATION BY REFERENCE OF MATERIAL SUBMITTED ELECTRONICALLY

This application claims priority to U.S. Provisional Patent Application No. 61/708,998, filed Oct. 2, 2012, the disclosure of which is hereby incorporated by reference in its entirety.

This application containing, as a separate part of disclosure, a Sequence Listing in computer-readable form (filename: 40000A_SeqListing.txt, created Oct. 2, 2013; 20,046 bytes), which is incorporated by reference in its entirety.

### TECHNICAL FIELD OF THE INVENTION

The invention relates to microorganisms engineered to produce increased amounts of organic acids and methods of use.

### BACKGROUND OF THE INVENTION

Organic acids have potential for displacing petrochemically derived monomers in a range of industrial applications such as polymers, food, pharmaceuticals and cosmetics. The use of bio-based organic acids could decrease the reliance on fossil fuels and benefit the environment in terms of reduced carbon dioxide production.

### SUMMARY OF THE INVENTION

Disclosed are recombinant microorganisms for producing organic acids. The disclosed recombinant microorganisms express the enzymes glucose-6-phophate-1-dehydrogenase and malate dehydrogenase, which results in increased organic acid production.

The following numbered paragraphs each succinctly define one or more exemplary variations of the invention:

1. A recombinant microorganism expressing glucose-6-phosphate dehydrogenase and malate dehydrogenase enzymes.

2. The recombinant microorganism of paragraph 1, wherein the recombinant organism is a succinic acid producing microorganism.

3. The recombinant microorganism of paragraph 1, comprising a 16S ribosomal RNA sequence with at least 90% identity to the 16S ribosomal RNA sequence of Actinobacillus succinogenes.

4. The recombinant microorganism of paragraph 1, which is Actinobacillus succinogenes, Bisgaard Taxon 6 and Bisgaard Taxon 10.

5. The recombinant microorganism of any one of paragraphs 1-4, comprising a heterologous or overexpressed polynucleotide encoding a glucose-6-phosphate dehydrogenase enzyme and a heterologous or overexpressed polynucleotide encoding a malate dehydrogenase enzyme.

6. The recombinant microorganism of any one of paragraphs 1-5, wherein the glucose-6-phosphate dehydrogenase or malate dehydrogenase are A. succinogenes enzymes.

7. The recombinant microorganism of any one of paragraphs 1-5, wherein the glucose-6-phosphate dehydrogenase and malate dehydrogenase is an A. succinogenes enzyme.

8. The recombinant microorganism of any one of paragraphs 1-6, wherein the glucose-6-phosphate dehydrogenase or malate dehydrogenase are E. coli enzymes.

9. The recombinant microorganism of any one of paragraphs 1-5, wherein the glucose-6-phosphate dehydrogenase and malate dehydrogenase is an E. coli enzyme.

10. The recombinant microorganism of any one of paragraphs 1-5, wherein the glucose-6-phosphate dehydrogenase comprises an amino acid sequence having at least about 40% sequence identity to the amino acid sequence set forth in SEQ ID NO: 2.

11. The recombinant microorganism of any one of paragraphs 1-5, wherein the malate dehydrogenase comprises an amino acid sequence having at least about 60% sequence identity to the amino acid sequence set forth in SEQ ID NO: 4.

12. The recombinant microorganism of any one of paragraphs 1-11, wherein the microorganism produces an increased amount of organic acid (e.g., succinic acid) compared to a microorganism expressing glucose-6-phosphate dehydrogenase or malate dehydrogenase enzymes alone.

13. The recombinant microorganism of any one of paragraphs 1-12, wherein the microorganism is capable of producing succinic acid at concentrations of about 50 g/L to 150 g/L.

14. A process for organic acid production, which process comprises culturing the recombinant microorganism of any one of paragraphs 1-13 in a fermentation medium.

15. A method of producing organic acid comprising the step culturing a recombinant microorganism with a carbon source under conditions favoring organic acid production, wherein the recombinant microorganism expresses at least one glucose-6-phosphate dehydrogenase and malate dehydrogenase enzymes.

16. The method of paragraph 15, wherein the recombinant microorganism expresses both glucose-6-phosphate dehydrogenase and malate dehydrogenase enzymes.

17. A method of producing organic acid comprising culturing the recombinant microorganism of any one of paragraphs 1-13 with a carbon source under conditions favoring organic acid production.

18. The method of any one of paragraphs 15-17, wherein the organic acid is succinic acid or propionic acid.

19. The method of paragraph 18, wherein the organic acid is succinic acid.

20. The method of any one of paragraphs 15-19, wherein the carbon source is glucose or sorbitol.

### BRIEF DESCRIPTION OF THE DRAWINGS

FIG. 1 depicts a schematic map of pL88 vector.

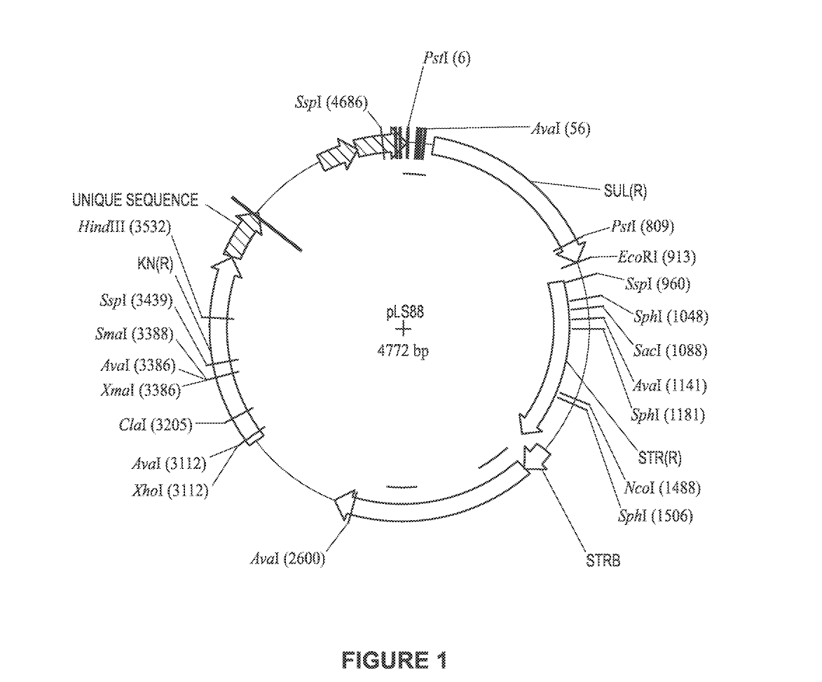

FIG. 2 depicts a schematic map of pISTONS1.

FIG. 3 depicts a schematic map of p830.60.

FIG. 4 depicts a schematic map of p856.78.

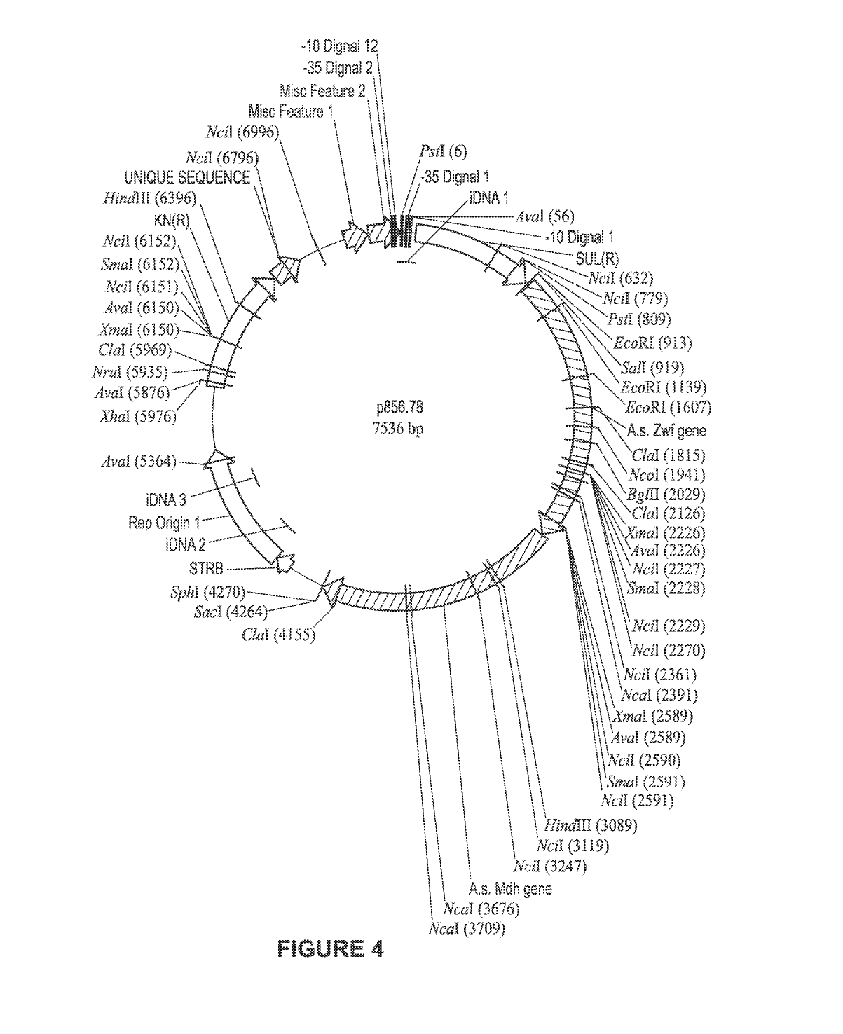

FIG. 5 is a sequence comparison between the amino acid sequences of the Zwf enzymes from E. coli (“query”) and A. succinogenes (“sbjct”).

FIG. 6 is a sequence comparison between the amino acid sequences of the Mdh enzymes from E. coli (“query”) and A. succinogenes (“sbjct”).

FIG. 7 is a sequence comparison between the amino acid sequences of the Mdh enzyme from Aspergillus flavus (“query”) and A. succinogenes (“sbjct”).

FIG. 8 is an A. succinogenes zwf nucleic acid sequence in p830.60.

FIG. 9 is an A. succinogenes mdh nucleic acid sequence in pISTONS1.

### DETAILED DESCRIPTION OF THE INVENTION

In the following detailed description, embodiments are described in sufficient detail to enable those skilled in the art to practice them, and it is to be understood that other embodiments may be utilized and that chemical and procedural changes may be made without departing from the spirit and scope of the present subject matter. The following detailed description is, therefore, not to be taken in a limiting sense, and the scope of embodiments is defined only by the appended claims.

The term “microorganism” refers to a single cell organism such as bacteria, fungi or yeast.

The term “recombinant microorganism” refers to a microorganism altered, modified or engineered (e.g., genetically engineered) such that it exhibits an altered, modified or different genotype or phenotype (e.g., when the genetic modification affects coding nucleic acid sequences of the microorganism) as compared to the naturally-occurring or starting microorganism from which it was derived.

Genetic manipulation can include, but is not limited to, altering or modifying regulatory sequences or sites associated with expression of a particular gene (e.g., by using strong promoters, inducible promoters or multiple promoters); modifying the chromosomal location of a particular gene; altering nucleic acid sequences adjacent to a particular gene, such as a sequence in the promoter region including regulatory sequences important for the promoter activity, a ribosome binding site, or transcription terminator; increasing the copy number of a particular gene; modifying proteins (e.g., regulatory proteins, suppressors, enhancers, transcriptional activators, and the like) involved in transcription or translation of a particular gene product; or any other conventional means of increasing expression of a particular gene.

As used herein, an “organic acid” includes an acid comprising at least one carboxylic group. Optionally, the organic acid is a C₁-C₁₀ organic acid, also optionally the organic acid comprises two carboxylate groups. For example, “organic acid” includes succinic acid or propionic acid. Other examples of organic acids include, but are not limited to, lactic acid, malic acid, citric acid, oxalic acid, and tartaric acid. As used herein, organic acids may also include the organic acid anion or a salt thereof. For example, “succinic acid” may be referred to as “succinate;” “propionic acid” may be referred to as “propionate.”

“Heterologous” refers to any polynucleotide or polypeptide that does not originate or is not native to the particular cell or organism where expression is desired.

“Overexpression” refers to expression of a polynucleotide to produce a product (e.g., a polypeptide or RNA) at a higher level than the polynucleotide is normally expressed in the host cell. An overexpressed polynucleotide is generally a polynucleotide native to the host cell, the product of which is generated in a greater amount than that normally found in the host cell. Overexpression is achieved by, for instance and without limitation, operably linking the polynucleotide to a different promoter than the polynucleotide's native promoter or introducing additional copies of the polynucleotide into the host cell.

A polypeptide or polynucleotide “derived from” an organism comprises the same amino acid sequence or nucleic acid sequence as the reference polypeptide or polynucleotide from the organism, or optionally contains one or more modifications to the native amino acid sequence or nucleotide sequence, as described further below, and optionally exhibits similar, if not better, activity compared to the native enzyme (e.g., at least 70%, at least 80%, at least 90%, at least 95%, at least 100%, or at least 110% the level of activity of the native enzyme). It will be appreciated that a polypeptide or polynucleotide “derived from” an organism is not limited to polypeptides or polynucleotides physically removed from a particular host organism.

Also herein, the recitations of numerical ranges by endpoints include all numbers subsumed within that range (e.g., 1 to 5 includes 1, 1.5, 2, 2.75, 3, 3.80, 4, 5, and the like). Furthermore, disclosure of a range includes disclosure of all subranges included within the broader range (e.g., 1 to 5 discloses 1 to 4, 1.5 to 4.5, 4 to 5, and the like).

The invention provides improved materials and methods for producing organic acid via fermentation. Fermentation performance is typically captured by three parameters: titer, productivity, and yield. The titer specifies the concentration of the product (e.g., organic acid, such as succinic acid) in the fermentation broth at the end of the fermentation. The productivity or rate of production represents the time to reach the titer of product. Yield is the quotient of mass of product per mass of feedstock (e.g., sugar, such as glucose). All three parameters affect the economics of a fermentative production process. A high titer facilitates product recovery; less water must be evaporated or removed, requiring less energy, which lowers the operating costs of the process. The productivity is linked to titer, adding a time dimension to it. High productivity implies a fast fermentation processes, and allows a reduction in fermentation vessel size, since the process can be run more often. Productivity affects mainly the capital cost of fermentative production processes. The yield affects the operating costs of the process—the higher the yield, the less raw material or feedstock is needed to obtain the product.

Titer, productivity, and yield are intertwined and affect one another. A fermentation that achieves a high titer in a short time will result in a high productivity and vice versa. The accumulation of a fermentation product or higher titer can slow production. Yield is inversely correlated to productivity, in that feedstock is used in the fermentation to support growth of the organism, multiplying of cells and building biomass, but also to metabolize feedstock into the desired product. Generally, more cells will produce more product and will produce it faster, which will result in higher productivity, but lower yield. Hence, fermentation optimization must balance all three parameters to achieve the most economical process. The ultimate goal is to achieve a process that has high values for all three parameters.

Remarkably, simultaneous over-production of enzymes in the pentose phosphate pathway (or Entner-Doudoroff pathway) and tri-carboxylic acid cycle in a recombinant microbe (e.g., Actinobacillus succinogenes) as described herein was discovered to promote a higher yield of succinic acid, which is a key factor in determining the economic viability of the fermentation process. In this regard, the invention is predicated, at least in part, on the surprising discovery that production of a glucose-6-phosphate dehydrogenase and a malate dehydrogenase in a microorganism results in increased organic acid production compared to the level of production achieved by a microorganism expressing a single enzyme alone. The pentose phosphate cycle (or Entner-Doudoroff pathway) utilizes several enzymes including glucose-6-phosphate dehydrogenase (e.g., glucose-6-phosphate-1-dehydrogenase, which is also referred to as Zwischenferment enzyme or Zwf); 6-phosphogluconolactonase; 6-phosphogluconate dehydrogenase, (also called Gnd); ribose-5-phosphate isomerase A and B; ribulose phosphate 3-epimerase; transketolase I and II; transaldolase A and B; 6-phosphogluconate dehydratase (Edd); and 2-keto-3-deoxyphosphogluconate aldolase (Eda). The tri-carboxylic acid cycle used in the anaerobic production of succinic acid uses, e.g., the enzymes phosphoenolpyruvate-carboxykinase, malate dehydrogenase, fumarase, and fumarate-reductase.

In one aspect, the invention provides a recombinant microorganism that produces both a glucose-6-phosphate dehydrogenase (e.g., Zwf) and a malate dehydrogenase (e.g., Mdh) such that, in various embodiments, the recombinant microorganism exhibits enhanced organic acid (e.g., succinic acid or propionic acid) production compared to an unmodified parent microorganism. The activities of glucose-6-phosphate dehydrogenase (EC 1.1.1.49) and malate dehydrogenase (EC 1.1.1.37) are well understood in the art. Glucose-6-phosphate dehydrogenase catalyzes, for example, the reaction D-glucose-6-phosphate+NADP+=>6-phospho-D-glucono-1,5-lactone+NADPH. Malate dehydrogenase converts oxalo-acetate to malate, or oxaloacetic acid to malic acid, reducing the substrate through the use of co-factors such as, but not limited to, NADH or NADPH.

Glucose-6-phosphate dehydrogenase and malate dehydrogenase are found in a wide variety of organisms. One or both of the enzymes may be native to the recombinant microorganism, but multiple copies of a polynucleotide encoding the enzyme are introduced into the host cell to increase production of the enzyme. In some embodiments, the recombinant microorganism expresses a native glucose 6-phosphate dehydrogenase and/or a native malate dehydrogenase at elevated levels (e.g., “overexpress” the enzyme) relative to levels present in non-recombinant microorganisms or the starting microorganism. Alternatively, one or both of the enzymes is not native to the recombinant microorganism. In various embodiments, the polynucleotides are derived from an organism that naturally produces succinic acid or malic acid. Depending on the embodiment of the invention, the polynucleotide is isolated from a natural source such as bacteria, algae, fungi, plants, or animals; produced via a semi-synthetic route (e.g., the nucleic acid sequence of a polynucleotide is codon-optimized for expression in a particular host cell, such as E. coli); or synthesized de novo.

Polynucleotides encoding malate dehydrogenase (e.g., mdh genes) may be derived, for example, from A. succinogenes (e.g., NC_{—}009655) or E. coli (EG10576 (EcoCyc)), or from other suitable sources shown to have the enzymatic activity. Similarly, the polynucleotide encoding the glucose 6-phosphate dehydrogenase is, in various embodiments, derived from E. coli. (e.g., EG11221 (EcoCyc); Accession Number: ECK1853) or A. succinogenes (e.g., GenBank Accession No. ABR73607.1 GI:150839636). Optionally, polynucleotides are codon optimized to facilitate expression in a particular microorganism. In some embodiments, the recombinant microorganism comprises a polynucleotide comprising the nucleic acid sequence of SEQ ID NO: 1, which encodes Zwf enzyme, and/or a polynucleotide comprising the nucleic acid sequence encoding SEQ ID NO: 3, which encodes an Mdh enzyme. Also contemplated herein are polynucleotides comprising a nucleic acid sequence that is at least 50%, at least 55%, at least 60%, at least 65%, at least 70%, at least 75%, at least 80%, at least 85%, at least 90%, or at least 95% (or any range of the foregoing percentages) to SEQ ID NO: 1 or SEQ ID NO: 3.

In some embodiments, the variant polynucleotide encodes a polypeptide having one or more biochemical activities of Zwf or Mdh enzyme. A variant polynucleotide may include, for example, a fragment of the zwf or mdh gene. In some embodiments, the recombinant microorganism may express a zwf or mdh gene expressed by A. succinogenes, E. coli, Basfia (e.g., Basfia succinicproducens), Mannheimia (e.g., Mannheimia succiniciproducens), Saccharomyces, Aspergillus or a variant thereof.

The inventors have discovered that expression of both Zwf and Mdh enzymes in a single fermentation process results in an increased organic acid production such as succinic acid or propionic acid. In some embodiments, a single recombinant organism may be used that co-expresses Zwf and Mdh. In other embodiments, the genes encoding the zwf and mdh are expressed in different organisms but the combined expression of both these enzymes in a single fermentation process is used in organic acid production. The invention contemplates a recombinant microorganism comprising (e.g., a microorganism that has been transformed with) a polynucleotide encoding a polypeptide having one or more biochemical activities of the Mdh enzyme, optionally in combination with a polynucleotide encoding a polypeptide having one or more biochemical activities of the Zwf enzyme, such as glucose-6-phosphate dehydrogenase activity and/or NADP reductase activity. For example, a suitable Zwf or Mdh enzyme is the E. coli Zwf or Mdh enzyme or a variant thereof. Put another way, in various embodiments, the glucose-6-phosphate dehydrogenase and/or malate dehydrogenase are/is E. coli or A. succinogenes enzymes.

A representative glucose 6-phosphate dehydrogenase amino acid sequence is provided in SEQ ID NO: 2, which is an A. succinogenes Zwf amino acid sequence. The recombinant microorganism may express a variant polypeptide having at least about 40% sequence identity to the amino acid sequence of a Zwf enzyme (e.g., SEQ ID NO: 2), and more desirably at least about 50%, at least about 60%, at least about 70%, at least about 80%, or at least about 90% sequence identity to the amino acid sequence of a Zwf enzyme (e.g., SEQ ID NO: 2). In various embodiments, the variant polypeptide comprises one or more conservative mutations with respect to a reference sequence, i.e., a substitution of an amino acid with a functionally similar amino acid. Put another way, a conservative substitution involves replacement of an amino acid residue with an amino acid residue having a similar side chain. Families of amino acid residues having similar side chains have been defined within the art, and include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine) and aromatic side chains (e.g., tyrosine, phenylalanine, tryptophan, and histidine).

A representative malate dehydrogenase amino acid sequence is provided in SEQ ID NO: 4, which is an A. succinogenes Mdh amino acid sequence. The recombinant microorganism may express a variant polypeptide having at least about 50% or at least about 60% sequence identity to the amino acid sequence of a Mdh enzyme (e.g., SEQ ID NO: 4), and more desirably at least about 70% or 80% or 90%, sequence identity to the amino acid sequence of a Mdh enzyme (e.g., SEQ ID NO: 4).

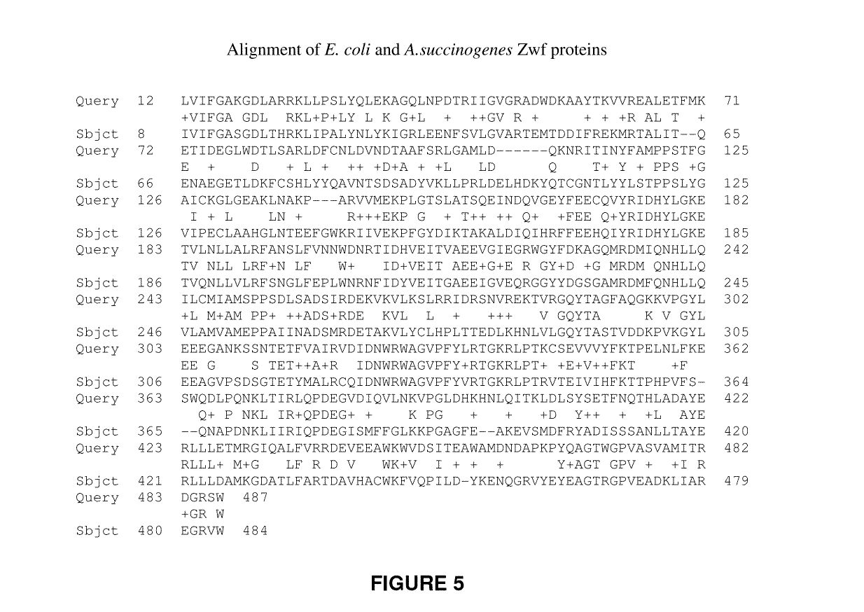

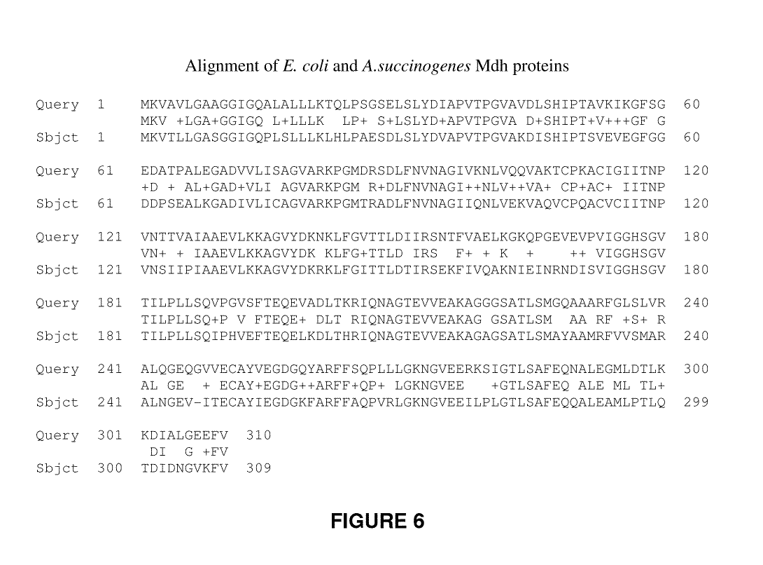

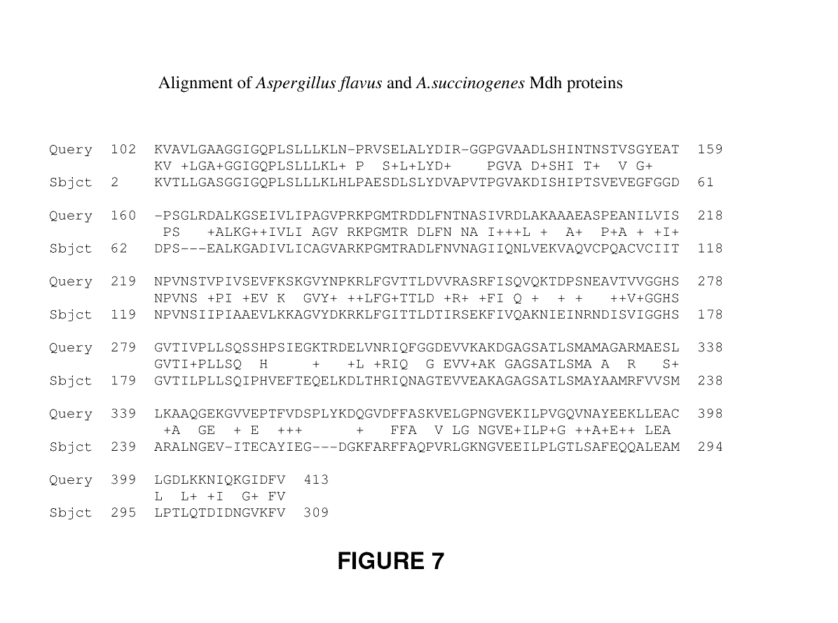

Substantial variation within the amino acid sequence is permitted in the context of the enzymes of the recombinant microorganism. The amino acid sequences of the Zwf enzymes from E. coli and A. succinogenes, for instance, share 44% identity, and both are suitable enzymes in the context of the invention. See FIG. 5. Similarly, the amino acid sequences of Mdh enzymes from E. coli and A. succinogenes share 69% identity, and both are suitable enzymes in the context of the invention. See FIG. 6. The Mdh enzyme is generally well conserved among different organisms. The amino acid sequence of the Mdh enzyme from Aspergillus flavus, a filamentous fungus, demonstrates about 50% identity to the Mdh enzyme sequence from A. succinogenes, and retains the ability to convert oxalo-acetate to malate. See FIG. 7.

Desirably, the variant polypeptide has one or more biochemical activities of the Zwf or Mdh enzyme, e.g., glucose-6-phosphate dehydrogenase and malate dehydrogenase activity. A variant polypeptide may include a fragment of the Zwf or Mdh enzyme. Methods of evaluating glucose-6-phosphate dehydrogenase activity and malate dehydrogenase activity are known in the art and available commercially from, e.g., Abcam, Cambridge, Mass., and Sigma Aldrich, St. Louis, Mo. An exemplary assay for determining glucose-6-phosphate dehydrogenase activity is described in Rowley and Wolf, J. Bacteriology, 173, 968-977 (1991) and Wolf et al., J. Bacteriology, 139, 1093-1096 (1979). Illustrative conditions for the reaction are as follows: a reaction mixture containing 50 mM Tris-HCl (pH 7.8), 10 mM MgCl₂, 1 mM NADP, 1 mM glucose-6-phosphate, and cell extract is prepared, and absorbance increase at 340 nm is measured. In various embodiments, the glucose-6-phosphate dehydrogenase encoded by the polynucleotide exhibits at least 70%, at least 75%, at least 80%, at least 85%, at least 90%, at least 95%, or 100% of the glucose-6-phosphate dehydrogenase activity of the polypeptide of SEQ ID NO: 2. The glucose-6-phosphate dehydrogenase encoded by the polynucleotide may exhibit greater than 100% of the glucose-6-phosphate dehydrogenase activity of the polypeptide of SEQ ID NO: 2 (e.g., 110% or more, 120% or, or 130% or more of the activity). Alternatively or in addition, the malate dehydrogenase produced by the recombinant microorganism exhibits at least 70%, at least 75%, at least 80%, at least 85%, at least 90%, at least 95%, or 100% of the malate dehydrogenase activity of the polypeptide of SEQ ID NO: 4. The malate dehydrogenase encoded by the polynucleotide may exhibit greater than 100% of the malate dehydrogenase activity of the polypeptide of SEQ ID NO: 4 (e.g., 110% or more, 120% or, or 130% or more of the activity). An exemplary assay for determining malate dehydrogenase activity is described in Samuelov, J. Bacteriology, 57, 3013 (1991). Illustrative conditions for the reaction are as follows: a reaction mixture containing 100 mM Tris-HCl, pH 8.1, 5 mM oxaloacetate, 0.3 mM NADH, 2 mM DTT, and cell extract is prepared; and absorbance decrease at 340 nm, representing oxidation of NADH, is measured.

The recombinant microorganism may be derived from any suitable microorganism such as, for instance, bacterial cells and other prokaryotic cells, and yeast cells. Typically, the microorganism is capable of producing an organic acid at a level suitable for commercial production. Suitable microorganisms for preparing recombinant microorganisms as described herein include succinic acid producing microorganisms. Exemplary microorganisms include, but are not limited to, organisms of the Pasteurellaceae family. Examples of Pasteurellaceae family members include members of the Actinobacillus genus, including A. succinogenes, Bisgaard Taxon 6 (deposited with the Culture Collection, University of Goteborg, Sweden (CCUG), under accession number 15568), Bisgaard Taxon 10 (deposited under CCUG accession number 15572), Mannheimia succiniciproducens, Basfia succinicproducens, E. coli, Anaerobiospirillum succiniciproducens, Ruminobacter amylophilus, Succinivibrio dextrinosolvens, Prevotella ruminicola, Ralstonia eutropha, and coryneform bacteria (e.g., Corynebacterium glutamicum, Corynebacterium ammoniagenes, Brevibacterium flavum, Brevibacterium lactofermentuin, Brevibacterium divaricatum); members of the Lactobacillus genus; yeast (e.g., members of the Saccharomyces genus such as Saccharomyces cerevisiae); and any subset thereof. In some embodiments, the recombinant strain may be (is) derived from a microorganism whose 16S rRNA has at least about 90% sequence identity (e.g., at least about 95% sequence identity) to 16S rRNA of Actinobacillus succinogenes. For example, the recombinant strain may be derived from a strain of Actinobacillus succinogenes, Bisgaard Taxon 6, or Bisgaard Taxon 10.

The recombinant microorganism may express a zwf gene, a mdh gene or both. The zwf or mdh genes may be native to the recombinant microorganism. In other embodiments, the zwf or mdh genes may be heterologous (i.e., not native to the microorganism from which the recombinant microorganism is derived). In some embodiments, the polynucleotide encoding the glucose-6-phosphate dehydrogenase and/or the polynucleotide encoding the malate dehydrogenase (e.g., the zwf or mdh gene) are optimized for expression in the recombinant microorganism. For example, zwf or mdh genes may be operationally linked to a promoter that facilitates gene overexpression relative to a non-recombinant microorganism. The promoter may be endogenous to the microorganism (e.g., native to the microorganism from which the recombinant microorganism is derived) or heterologous to the microorganism. In some embodiments, the zwf and mdh genes are from A. succinogenes and are regulated by their respective zwf and mdh promoters also from A. succinogenes.

The polynucleotide encoding the glucose-6-phosphate dehydrogenase and/or the polynucleotide encoding the malate dehydrogenase (e.g., zwf or mdh genes) are optionally modified to facilitate translation of the corresponding mRNA. For example, a zwf or mdh gene may be modified to include codons that are not present in the endogenous or native gene. These non-endogenous codons may be selected to reflect the codon usage frequency in the recombinant microorganism. Codon usage tables have been developed for many microorganisms and are known in the art. For example, the polynucleotide encoding the glucose-6-phosphate dehydrogenase and/or the polynucleotide encoding the malate dehydrogenase (e.g., zwf or mdh genes) may be modified to reflect the codon usage frequency for A. succinogenes as provided in U.S. Pat. No. 8,119,377, which patent is incorporated by reference in its entirety.

The recombinant microorganism may be derived from a strain that produces high levels of one or more organic acids such as succinic acid or propionic acid, or the recombinant microorganism may be selected or engineered to produce high or enhanced levels of one or more organic acids such as succinic acid or propionic acid relative to a non-recombinant microorganism.

Optionally, the recombinant microorganism is selected or engineered (or both) to be resistant to relatively high levels of undesirable by-products or to produce relatively low levels of undesirable by-products. For example, after introduction of the polynucleotides encoding the glucose-6-phosphate dehydrogenase and malate dehydrogenase into the cell (e.g., transformation), a population of recombinant microorganisms is optionally grown in the presence of sodium monofluoroacetate to select strains that are resistant to relatively high acetate levels or strains that produce relatively low acetate levels. Undesirable by-products include, but are not limited to, formate (or formic acid), acetate (or acetic acid), and/or pyruvate (or pyruvic acid), lactate, 1,3-propanediol, and ethanol. Methods for selecting strains that produce low acetate levels are known in the art. See, e.g., U.S. Pat. No. 5,521,075 and U.S. Pat. No. 5,573,931, which are incorporated herein by reference, particularly with respect to their discussion of microbial strain selection. For example, strains of microorganisms that produce relatively low acetate levels may be selected by growing the microorganisms in the presence of a toxic acetate derivative, such as sodium monofluoroacetate at a concentration of about 1.0 to about 8.0 g/L. Selected strains may produce relatively low acetate levels (e.g., less than about 10 g/L, less than about 7 g/L, less than about 5 g/L, or less than about 2.0 g/L), formate (e.g., less than about 2.0 g/L), and/or pyruvate (e.g., less than about 3.0 g/L) in a glucose fermentation. One suitable monofluoroacetate resistant strain for producing a recombinant microorganism or derivative is a strain of A. succinogenes deposited under ATCC accession number 55618. See also U.S. Pat. No. 5,573,931, which describes suitable methods for preparing microbial strains that are resistant to monofluoroacetate. Other suitable monofluoroacetate resistant strain examples include FZ45 and FZ53, also described in U.S. Pat. No. 5,573,931.

A polynucleotide encoding a glucose-6-phosphate dehydrogenase and a polynucleotide encoding a malate dehydrogenase (or a polypeptide with one or more biochemical activities of the Zwf or Mdh enzyme) may be obtained by employing methods known in the art (e.g., PCR amplification with suitable primers and cloning into a suitable DNA vector). The polynucleotide sequences of suitable zwf genes encoding glucose-6-phosphate dehydrogenase activity have been disclosed. (See, e.g., GenBank). For example, the polynucleotide sequence of the A. succinogenes zwf gene has been published in U.S. Pat. No. 8,119,377, which is hereby incorporated by reference. See also Joint Genome Institute, Department of Energy website, NCBI accession number NC_{—}009655. The E. coli zwf gene is deposited with GenBank (e.g., under GenBank Accession Number NC 000913 and GenBank Accession Number M55005). The zwf gene or variants thereof may be obtained by PCR amplification of a microorganism's genomic DNA with appropriate primers.

The mdh sequences may be obtained, for example, from Actinobacillus succinogenes, GenBank NC_{—}009655, or E. coli. EG10576 (EcoCyc).

The DNA vector may be any suitable vector for expressing polynucleotide(s) in a recombinant microorganism. Suitable vectors include plasmids, artificial chromosomes (e.g., bacterial artificial chromosomes), and/or modified bacteriophages (e.g., phagemids). The vector may be designed to exist as an epigenetic element and/or the vector may be designed to recombine with the microorganism's genome.

The polynucleotide typically includes a promoter operationally linked to a nucleic acid sequence that encodes a glucose-6-phosphate dehydrogenase or malate dehydrogenase (i.e., a polypeptide having Zwf or Mdh enzyme activity). The promoter may be endogenous or native to the microorganism from which the recombinant microorganism is derived or heterologous to the microorganism (e.g., derived from a source other than the recombinant microorganism). Furthermore, the promoter may be the native promoter for a selected glucose-6-phosphate dehydrogenase or malate dehydrogenase coding sequence (i.e., zwf or mdh gene) or may be a promoter other than the native promoter for a selected glucose-6-phosphate dehydrogenase or malate dehydrogenase (e.g., a non-zwf or mdh gene promoter). In some embodiments, the recombinant microorganism is a A. succinogenes strain, and the A. succinogenes zwf or mdh promoter is used. In other embodiments, the promoter may be an A. succinogenes phosphoenolpyruvate (PckA) carboxykinase promoter, deposited under GenBank accession number AY308832, including nucleotides 1-258, or a variant thereof, as disclosed in U.S. Pat. No. 8,119,377, which is hereby incorporated by reference. Any suitable promoter (e.g., inducible, constitutive, strong and the like) that can direct expression of the desired sequence in the desired microorganism may be used. The promoter may be operationally linked to the polynucleotide encoding glucose-6-phosphate dehydrogenase or polynucleotide encoding malate dehydrogenase (e.g., zwf or mdh gene) using cloning methods that are known in the art. For example, the promoter and the coding sequence (e.g. zwf and mdh gene) may be amplified by PCR using primers that include compatible restriction enzyme recognition sites. The amplified promoter and coding sequence then may be digested with the enzyme and cloned into an appropriate vector that includes a suitable multiple cloning site.

In addition, the polynucleotide(s) or vector may include or encode a selectable marker. The selectable marker may impart resistance to one or more antibiotic agents. For example, selectable markers may include genes for ampicillin resistance, streptomycin resistance, kanamycin resistance, tetracycline resistance, chloramphenicol resistance, sulfonamide resistance, or combinations of these markers. Typically, the selectable marker is operationally linked to a promoter that facilitates expression of the marker. Plasmids and other cloning vectors that include selectable markers are known in the art.

Any suitable host cell may be used to store or propagate a vector comprising the polynucleotide(s). The host cell optionally expresses a coding sequence on the DNA molecule. Suitable host cells also may include cells that are capable of producing (i.e., cells that produce) an organic acid in a fermentation process, such as succinic acid or propionic acid at a concentration suitable for commercial production (e.g., at least about 20 g/L, more suitably at least about 50 g/L, and more suitably at least about 100 g/L).

In the context of the invention, methods for producing an organic acid typically include fermenting a nutrient medium with a recombinant microorganism. In some embodiments the recombinant microorganism in the fermentation medium expresses a zwf and mdh gene. For example, the method may include fermenting a nutrient medium with a recombinant A. succinogenes that expresses a zwf and mdh gene (e.g., a native A. succinogenes zwf or mdh gene). It can be envisioned that other combinations of genes and organisms may be used to ferment medium to produce organic acid. For example a heterologous zwf gene and endogenous mdh gene may be both expressed from a heterologous promoter in a suitable organism or any other suitable combinations.

Thus, in one aspect, the invention provides a method of producing organic acid comprising culturing the recombinant microorganism described herein with a carbon source under conditions favoring organic acid production. The recombinant microorganism, for example, comprises a heterologous or overexpressed polynucleotide encoding a glucose-6-phosphate dehydrogenase enzyme and a heterologous or overexpressed polynucleotide encoding a malate dehydrogenase enzyme, and produces the enzymes to enable production of a desired organic acid. Organic acids produced in the fermentation may include, for example, succinic acid or propionic acid, or any other organic acid discussed herein.

One suitable recombinant microorganism for the methods described herein is a recombinant strain of A. succinogenes that expresses the E. coli zwf gene, deposited under ATCC accession number PTA-6255. This organism, in combination with another recombinant organism expressing the mdh gene, may be both grown on fermenting nutrient medium to produce organic acid. Alternatively, the strain deposited under ATCC accession number PTA-6255 is modified to overexpress mdh or produce heterologous Mdh. The methods also may include fermenting a nutrient medium with a recombinant strain of Bisgaard Taxon 6 or Bisgaard Taxon 10 that express a zwf or mdh or both genes (e.g., a heterologous zwf or mdh gene such as from E. coli) to produce succinic acid.

The nutrient medium typically includes a fermentable carbon source. The fermentable carbon source may be provided by a fermentable biomass. A fermentable biomass may be derived from a variety of crops and/or feedstocks including: sugar crops (e.g., sugar, beets, sweet sorghum, sugarcane, fodder beet); starch crops (e.g., grains such as corn, wheat, sorghum, barley, and tubers such as potatoes and sweet potatoes); cellulosic crops (e.g., corn stover, corn fiber, wheat straw, and forages such as Sudan grass forage, and sorghum). The biomass may be treated to facilitate release of fermentable carbon source (e.g., sugars). For example, the biomass may be treated with enzymes such as cellulase and/or xylanase, to release simple sugars, and/or may be treated with heat, steam, or acid to facilitate degradation. The fermentable carbon source may include simple sugars and sugar alcohols such as glucose, maltose, mannose, mannitol, sorbitol, galactose, xylose, xylitol, arabinose, arabitol, and mixtures thereof. In some embodiments, non-purified, minimally processed or crude carbon sources may be used for organic acid production. In one embodiment, the carbon source may include crude polyols described in U.S. Patent Application Ser. No. 60/709,036, entitled “Integrated Systems & Methods for Organic Acid Productions” filed on the same date as the U.S. Provisional Patent Application No. 61/708,998, to which the instant application claims priority, which application is incorporated by reference herein in its entirety. Crude polyols include, for example, polyols produced via, e.g., fermentation or hydrogenation, which are otherwise subjected to minimal processing. For example, crude polyols are not subjected to a filtration step to remove solids. As such, crude polyols are less pure than refined polyols.

The methods of the invention result in a relatively high yield of succinic acid relative to an input carbon source, such as sugar (e.g., glucose or sorbitol). For example, the methods optionally have a succinic acid yield (g) of at least about 70%, at least about 75%, at least about 80%, at least about 85%, at least about 90%, or at least about 95% (or any range of the foregoing percentages) relative to carbon source (e.g., glucose) input (g), or a propionic acid yield (g) of at least 56% (e.g., at least about 60%, at least about 65%, at least about 70%, at least about 75%, at least about 80%, at least about 85%, at least about 90%, or at least about 95%, or any range of the foregoing percentages) relative to carbon source (e.g., glucose) input (g). In some embodiments, the yield may be calculated as % succinic acid or propionic acid yield (mol)/carbon source (e.g., glucose) input (mol). As such, the methods may have a succinic acid or propionic yield (mol) of at least about 154% relative to carbon source (e.g., glucose) input (mol). Desirably, the methods may have a succinic acid or propionic acid yield (mol) of at least about 130% or at least about 170% relative to carbon source (e.g., glucose) input (mol).

The methods may result in a relatively high succinic acid concentration (e.g., relative to a method that uses a non-recombinant microorganism in a fermentation). For example, a fermentation may reach a concentration of at least about 50 g/L succinic acid (e.g., at least about 60 g/L, at least about 70 g/L, or at least about 80 g/L). Desirably, a fermentation may reach a concentration of at least about 90 g/L succinic acid (e.g., at least about 100 g/L succinic acid, at least about 110 g/L succinic acid, or at least about 120 g/L succinic acid) or more desirably, a concentration of at least about 130 g/L succinic acid and even more desirably a concentration of at least about 140 g/L. In some embodiments, the fermentation typically does not produce substantial levels of undesirable by-products such as acetate, formate, pyruvate, and mixtures thereof (e.g., no more than about 11.0 g/L acetate, no more than about 10.0 g/L acetate, no more than about 9.0 g/L acetate, no more than about 8.0 g/L acetate, no more than about 7.0 g/L acetate, no more than about 6.0 g/L acetate, no more than about 5.0 g/L acetate, no more than about 4.0 g/L acetate, no more than about 3.0 g/L, or no more than about 2.0 g/L acetate (e.g., 1.5 g/L or less or 1.0 g/L or less); no more than about 2.0 g/L formate (e.g., 1.5 g/L or less or 1.0 g/L or less); and/or no more than about 3.0 g/L pyruvate (e.g., 2.5 g/L or less or 2.0 g/L or less or 1.5 g/L or less or 1.0 g/L or less)).

The method described herein optionally results in relatively high production rates. In various embodiments, the method achieves a productivity of at least about 1.0 g/L-h, at least about 1.5 g/L-h, at least about 2.0 g/L-h, at least about 2.5 g/L-h, at least about 3.0 g/L-h, at least about 3.5 g/L-h, or at least about 4.0 g/L-h.

In one embodiment, the recombinant microorganism is Actinobacillus succinogenes that expresses a heterologous zwf and mdh gene. The heterologous zwf and mdh genes may be optimized for expression in Actinobacillus succinogenes. The heterologous zwf and mdh genes may be encoded by E. coli. For example, a recombinant organism is Actinobacillus succinogenes deposited under ATCC Accession Number PTA-6255 (American Type Culture Collection, Manassas, Va., USA), which is optionally modified to overproduce Mdh. An alternative recombinant microorganism is an A. succinogenes strain deposited under ATCC Accession Number PTA-120462, which is a strain that produces increased levels of Zwf and Mdh compared to a parent (unmodified) A. succinogenes strain. The recombinant strain may be capable of producing succinic acid or propionic acid at concentrations of about 50 g/L to about 150 g/L (e.g., in a fermentation system that utilizes a suitable carbon source). The recombinant strain may be resistant to levels of sodium monofluoroacetate of at least about 1 g/L.

In another embodiment, the recombinant strain is Actinobacillus succinogenes, which includes a DNA molecule (i.e., a polynucleotide) comprising a transcription promoter for Actinobacillus succinogenes operationally linked to a heterologous zwf or mdh gene (e.g., a A. succinogenes promoter linked to a Zwf coding sequence and a A. succinogenes promoter linked to a Mdh coding sequence). The transcription promoter may include the A. succinogenes phosphoenolpyruvate (PckA) carboxykinase promoter or a variant thereof, whose sequence is disclosed in U.S. Pat. No. 8,119,377. The heterologous zwf gene may encode E. coli Zwischenferment enzyme or a variant thereof. The heterologous mdh gene may encode E. coli Mdh enzyme or a variant thereof. Optionally, the zwf gene and/or the mdh gene may be optimized for expression in Actinobacillus succinogenes. The DNA molecule may be epigenetic (e.g., present on a plasmid). The DNA molecule may include a selectable marker (e.g., kanamycin resistance, ampicillin resistance, streptomycin resistance, sulfonamide resistance, tetracycline resistance, chloramphenicol resistance, or a combination thereof).

In some embodiments, the DNA molecule (polynucleotide) comprises a transcription promoter for a succinic acid producing microorganism operationally linked to a heterologous zwf gene. The transcription promoter may include a zwf promoter. The DNA molecule may be present within a plasmid. The DNA molecule may be present in a host cell (e.g., a host cell that produces succinic acid or propionic acid at concentrations of about 50 g/L to about 150 g/L).

In one embodiment, the method for producing succinic acid is provided. The method comprises fermenting a nutrient medium with a recombinant microorganism that expresses a heterologous zwf gene to produce a glucose-6-phosphate dehydrogenase enzyme, optionally in combination with a second recombinant microorganism expressing a heterologous mdh gene to produce a malate dehydrogenase enzyme. In various embodiments, the recombinant microorganism expresses a heterologous mdh gene and a heterologous zwf gene. The recombinant microorganism may include a recombinant strain of Actinobacillus succinogenes (e.g., A. succinogenes recombinant strain deposited under ATCC Accession Number PTA-6255). The recombinant microorganism may include a recombinant strain of Bisgaard Taxon 6, Bisgaard Taxon 10 and the like. The heterologous zwf gene may include the E. coli zwf gene. The heterologous mdh gene may include the E. coli mdh gene. Optionally, the recombinant strain is resistant to levels of sodium monofluoroacetate of at least about 1 g/L. Optionally, the recombinant strain is capable of producing succinic acid or propionic acid at concentrations of about 50 g/L to about 150 g/L. The nutrient medium may include a fermentable sugar (e.g., glucose). Typically, the method results in a succinic acid yield (g) of at least about 100% relative to glucose (g).

In another embodiment, the recombinant microorganism is a recombinant strain of a succinic acid producing microorganism. The recombinant microorganism comprises (e.g., has been transformed with) a heterologous zwf gene (i.e., a heterologous polynucleotide encoding a glucose-6-phosphate dehydrogenase). The heterologous zwf gene may be optimized for expression in the microorganism. In some embodiments, the heterologous zwf gene may encode E. coli Zwf enzyme.

In another embodiment, the recombinant microorganism is a recombinant strain of a succinic acid producing microorganism that has been transformed with a DNA molecule (a polynucleotide) that includes a transcription promoter for phosphoenolpyruvate (PckA) carboxykinase operationally linked to polynucleotide encoding a polypeptide having Zwf enzyme activity. The recombinant microorganism optionally has been transformed with a DNA molecule (a polynucleotide) that includes a transcription promoter for phosphoenolpyruvate (PckA) carboxykinase operationally linked to polynucleotide encoding a polypeptide having Mdh enzyme activity. The transcription promoter may include the Actinobacillus succinogenes phosphoenolpyruvate (PckA) carboxykinase promoter. In another embodiment, the recombinant microorganism is a recombinant strain transformed with a DNA molecule that is epigenetic. The DNA molecule may be present on a plasmid.

In another embodiment, the recombinant microorganism is a recombinant strain that is capable of producing succinic acid or propionic acid at concentrations of about 50 g/L to about 150 g/L.

The recombinant strain may be resistant to levels of sodium monofluoroacetate of at least about 1 g/L. In some embodiments, the recombinant strain is produced using recombinant Actinobacillus succinogenes deposited under ATCC Accession Number PTA-6255, or the recombinant strain is Actinobacillus succinogenes deposited under ATCC Accession Number PTA-120462.

In another embodiment, the recombinant microorganism is used for producing succinic acid or propionic acid in a method that includes fermenting a nutrient medium with the recombinant microorganism. The nutrient medium typically includes fermentable sugar such as glucose or sorbitol. The method may result in a succinic acid yield (g) of at least about 70%, at least about 80%, at least about 90%, or at least about 100% relative to glucose (g). The method may result in a propionic acid (g) of at least about 62% to about 72% relative to glucose (g), or at least about 60%, or at least about 70%, or at least about 80%, or at least about 90% (or any range within these percentages).

The methods for producing an organic acid can include growing suitable microorganisms in a suitable fermentation broth which contains a carbon source (e.g., crude sorbitol). In one embodiment, the fermentation broth also contains a nitrogen source, inorganic salts, vitamins or growth promoting factors, and the like. In some embodiments, the salts, ammonium source and other nutrient media requirements may be obtained from corn steep liquor (CSL), a by-product of the corn wet-milling industry. With respect to salts, sodium sources include, but are not limited to, Na₃PO₄, Na₂HPO₄, NaH₂PO₄, Na₂CO₃, NaHCO₃, NaCl, and Na from organic salts (e.g. monosodium glutamate, sodium acetate). The sodium concentration can range between about 1200 mg/l to about 6800 mg/L. In some embodiments, the sodium concentration is from about 3000 mg/l to about 3500 mg/L. The invention is not limited to sodium salts; other salts are contemplated as part of the invention.

Fermentations can be conducted by combining the carbon source and fermentation broth in any suitable fermentor, and inoculating with a suitable microorganism. Fermentation may be carried out either aerobically or anaerobically under conditions conducive to the growth of the microorganism and production of the suitable organic acid. In one embodiment, fermentation temperature is maintained within the range of at least about 25° C., and less than about 50° C. In some embodiments, the temperature is between about 30° C. and about 39° C. (e.g., about 38° C.). In some embodiments, the temperature is between about 30° C. and about 37° C.

The fermentation broth may also include a betaine or an addition salt or a mixture thereof. In some embodiments the betaine is betaine-HCl, betaine free base or the like.

In other embodiments, the betaine may be present as a component of a feed product which contains betaine. Exemplary betaines or at least one feed product which contains betaine include betaine, amino acid fermentation byproduct solubles, molasses containing betaine, condensed separator byproduct, condensed molasses solubles, vinasse, or any mixture thereof. Other examples of feed products which contain betaine include a condensed, extracted glutamic acid fermentation product, amino acid fermentation byproduct solubles from the fermentative production lysine, amino acid fermentation byproduct solubles from the fermentative production threonine, or amino acid fermentation byproduct solubles from the fermentative production tryptophan. The betaine concentration may range from about 0.05 g/l to about 2 g/l. In some embodiments the betaine concentration used may be 0.2 g/l to 0.5 g/l.

In one embodiment, the pH of the fermentation broth at the beginning of fermentation is within the range of about pH 6-7 or the range of about pH 7 to about pH 8.0 (e.g., about pH 8.0). Fermentation pH can be controlled by addition of base, e.g., pH may be controlled to achieve about pH 5 to about pH 7, or about pH 6 to about pH 7 (e.g., about pH 6.6), or about pH 4.5 to about 6, or from about 5 to about 5.5, as the fermentation progresses. Magnesium bases are suitable for controlling pH in the context of the invention. In one embodiment, MgCO₃ is provided to control the pH in a CO₂ atmosphere to optionally achieve an approximate pH of about pH 6.0 to about pH 7.0 (e.g., about pH 6.4 to about pH 6.8), preferably about pH 6.6. Mg(OH)₂ also is appropriate. NH₄OH, NaOH, gaseous NH₃, Na₃PO₄, or their carbonate forms may be used, although the method is not dependent on a particular pH control agent.

### EXAMPLES

### Example 1

### Microorganism Strains and Plasmids

A. succinogenes strains FZ45 and FZ53 are stable bacterial variants of Actinobacillus succinogenes 130Z, which is resistant to sodium monofluoroacetate. See Guettler et al., INT'L J. SYST. BACT. (1999) 49:207-216; and U.S. Pat. No. 5,573,931. An E. coli-A. succinogenes shuttle vector pLS88 (deposited at the American Type Culture Collection as ATCC accession no. 86980) was obtained from Dr. Leslie Slaney, University of Manitoba, Canada. Plasmid pLS88 is described as having been isolated from Haemophilus ducreyi and may confer resistance to sulfonamides, streptomycin, and kanamycin.

### Genetic Manipulations:

Recombinant DNA manipulations generally followed methods described in the art. Plasmid DNA was prepared by the alkali lysis method. Typical resuspension volumes for multicopy plasmids extracted from 1.5 ml cultures were 50 μl. Larger DNA preparation used the Qiagen Plasmid Purification Midi and Maxi kit according to the manufacturer's instructions. Restriction endonucleases, molecular weight standards, and pre-stained markers were purchased from New England Biolabs and Invitrogen and digests were performed as recommended by the manufacturers, except that an approximately 5-fold enzyme excess was used. DNA was analyzed on Tris-acetate-agarose gels in the presence of ethidium bromide. DNA was extracted from agarose gels and purified using the Qiagen gel extraction kit according to the manufacturer's instructions. DNA was dephosphorylated using shrimp or calf alkaline phosphatase (Roche) in combination with restriction digests. The phosphatase was heat inactivated at 70° C. for 15 min or by passing through a Qiagen purification column. Ligations were performed using a 3- to 5-fold molar excess of insert to vector DNA in a 20 μl reaction volume and 1 μl of T4 DNA Ligase (New England Biolabs) for 1 hour at 25° C. E. coli transformation was performed by using “library efficiency competent cells” purchased from Invitrogen, following the manufacturer's instructions.

Transformations using ligation mixes were plated without dilutions on standard LB plates containing the appropriate antibiotic. PCR amplifications were carried out using the Perkin Elmer manual as a guideline. Primer designs were based on published sequences (as provided the National Center for Biotechnology Information (NCBI) database) and obtained from Invitrogen Life Sciences. The primers included engineered restriction enzyme recognition sites. Primers were analyzed for dimer and hairpin formation and melting temperature using the Vector NTI program. All primers were ordered from the Michigan State Macromolecular Structure Facility. PCR amplifications were carried out in an Eppendorf Gradient Master Cycler, or in a Perkin Elmer Thermocycler. Starting annealing temperatures were determined using the Vector NTI program for each primer pair. Restriction enzymes for digesting the amplified products were purchased from Invitrogen or New England Biolabs.

A. succinogenes competent cells for electroporation were prepared by growing cells in the presence of MgCO₃ in tryptic soy broth, TSB, medium supplemented with glucose and harvested in early to mid log-phase. Unused carbonate was removed by low speed centrifugation. Cells were spun down, washed twice with sterile water, twice with 10% v/v glycerol, and resuspended in 0.01× the original culture volume of 10% glycerol. Cells were suspended into small aliquots, flash frozen and stored at −80° C. 40 μl of prepared cells were used for electroporation, using 0.1 cm cuvettes and a BioRad GenePulser with settings of 400 W, 25 mF, 1.8 kV. Following electroporation, 1 ml room temperature TSB medium was immediately added to the cuvette and incubated at 37° C. for 1 h. The cell solution was plated on TSB agar plates containing the appropriate antibiotic, and incubated for 3 days at 37° C. in a CO₂ atmosphere.

Plasmid p830.60

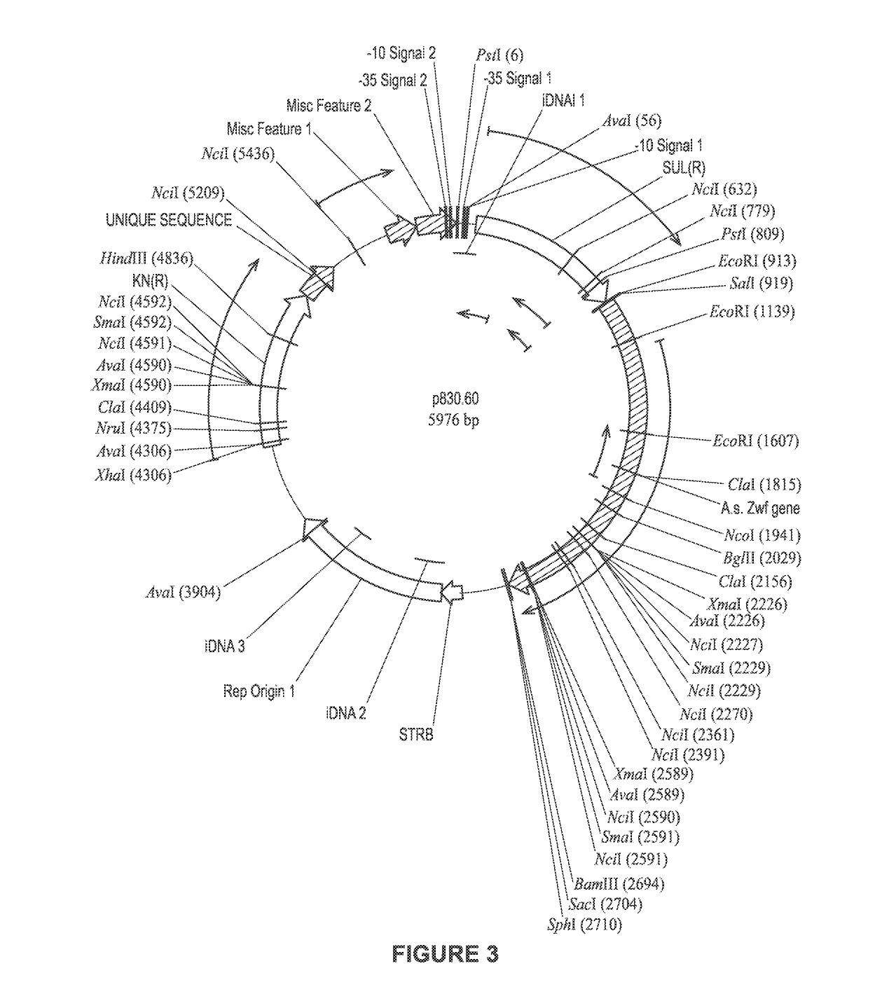

The A. succinogenes zwf gene and promoter were amplified from Actinobacillus succinogenes FZ45 genomic DNA, using primer TD299 (SEQ ID No. 5), and TD300, (SEQ ID No. 6), and inserted into the BamH1 and Sal1 sites of pJR762.47, a derivative of pLS88, with a multicloning site, inserted into the EcoRI and SphI sites of pLS88 and shown as FIG. 3.

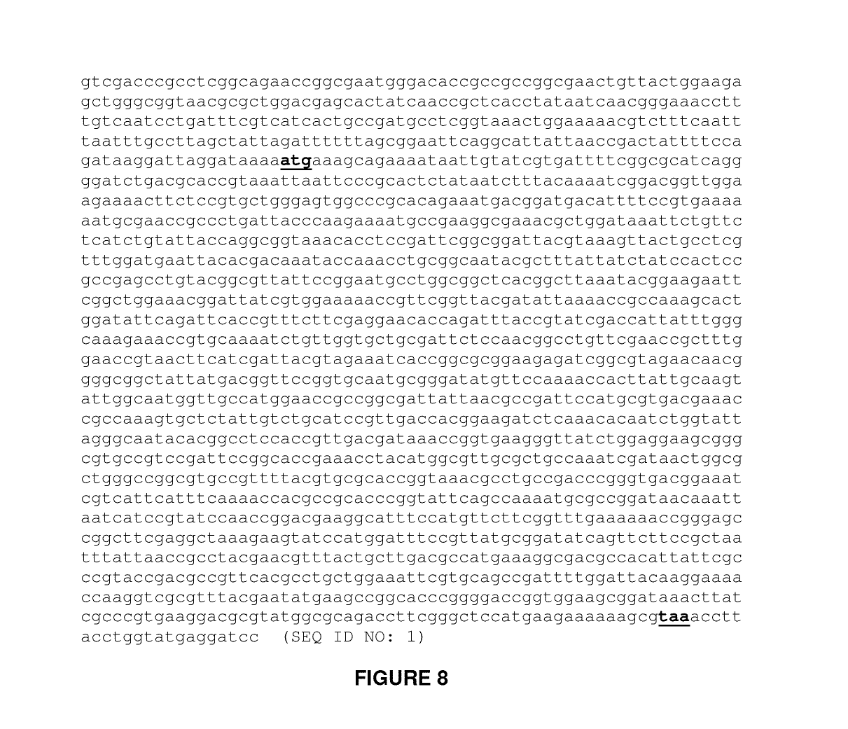

The cloned gene in p830.60 is 1781 base pairs (bp), the coding region starts at position 272 (bold underlined atg in FIG. 8) and stops at 1757 (bold underlined taa in FIG. 8) and its sequence is provided as SEQ ID NO:1. The promoter sequence begins at the start and ends before the coding sequence.

Plasmid pISTONS1

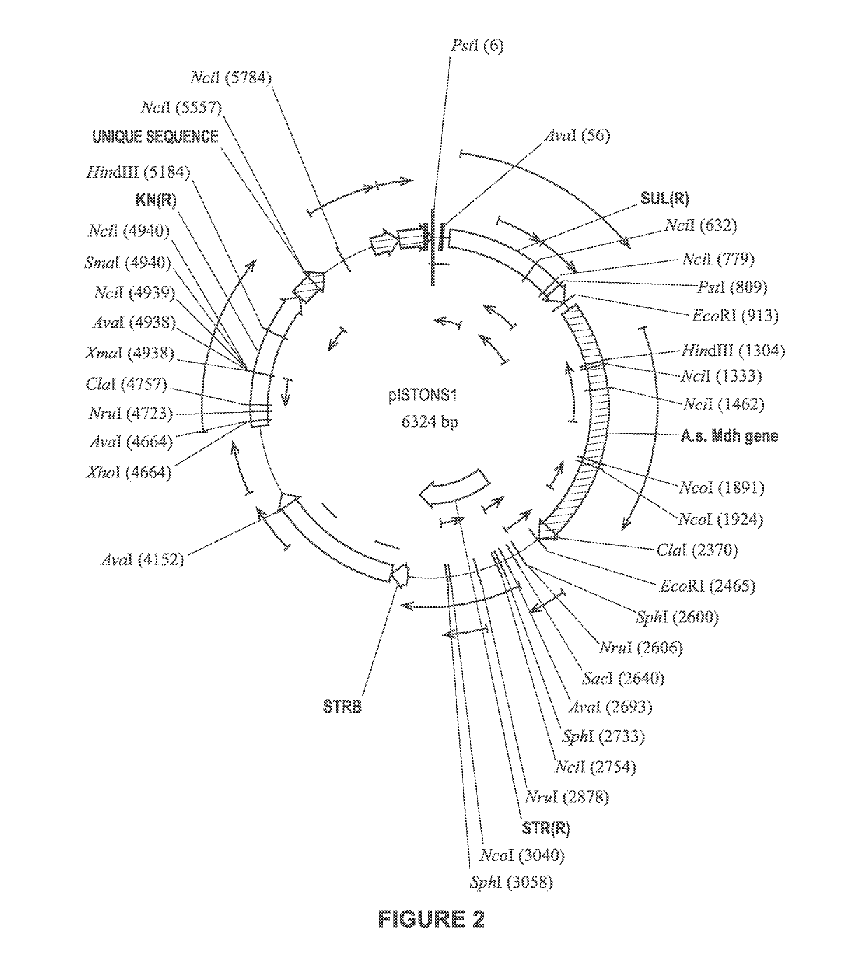

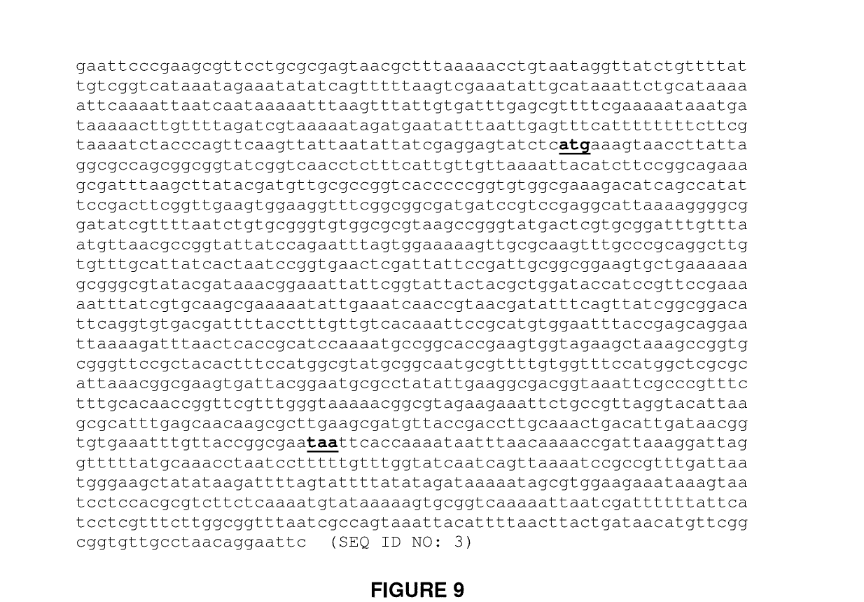

The A. succinogenes Mdh gene and promoter were amplified from Actinobacillus succinogenes FZ45 genomic DNA using primers TD223 (SEQ ID NO: 7), and TD218 (SEQ ID NO: 8). The amplified DNA was cloned as an EcoRI fragment and inserted into the EcoRI site of pLS88 as shown in FIG. 2. The mdh gene in pISTONS1 is 1558 bp, with the coding region starting at 303 (bold underlined atg in FIG. 9) and ending at 1239 (bold underlined taa in FIG. 9) and provided as SEQ ID NO: 3 and the promoter is shown from the start of the sequence and ends before the coding region.

Plasmid p856.78

The A. succinogenes mdh gene was excised from pISTONS1 using the restriction enzyme EcoRI, the ends were filled in with Klenow Polymerase to prepare blunt ends and ligated to plasmid p830.60, which had been linearized with the restriction endonuclease BamHI and the ends had been filled in with Klenow Polymerase. The two genes are arranged as head-to-tail insertions.

### Example 2

### Succinic Acid Production from Sorbitol by Overexpression of Zwf and Mdh Enzymes

Actinobacillus succinogenes strains (FZ53; FZ53/pLS88; FZ53/p830.60; FZ53/pPISTONS1 and FZ53/p856.78) were cultivated in fermentation vessels (Bioflo III, New Brunswick) containing 2 L of culture medium. The culture medium contained 120 g/L sorbitol, 30 g/L (solids), corn steep liquid (CSL) (10-12% solids from ADM), 1.6 g/L Mg(OH)₂, 0.2 mg/L biotin, 0.5 g/L betaine HCl, 0.2 mg/L MSG, 6.5 mM sodium phosphate, 7 g/L Na2CO3, 0.5 g/L yeast extract (AG900).

The fermentor was inoculated with 6.25% inoculum from a vial culture cultivated in the same medium as the fermentor (but omitting the Mg(OH)₂) and incubated with constant shaking at 150 rpm at 38° C. for 13 h.

Inoculated fermentors were incubated at 38° C. with agitation at 380 rev/min and a sparge of 0.025 to 0.05 vvm CO₂. The pH of the fermentation medium was maintained at pH 6.8 by automatic addition of 6 M Mg(OH)₂ or addition of MgCO₃ at 120 g/L.

A carbon source feed was implemented between 12 and 22 h during which 15 g of additional carbon source were added to the fermentation vessel.

### Sugar and Organic Acid Determination:

Samples were removed from the fermentors at intervals and the solids removed by centrifugation at 10,000×g for 4 min. The supernatant was filtered through a 0.2 μM filter prior to analysis. Sugar and organic acid concentrations in the culture supernatants and filtrates were determined by HPLC (Agilent 1200 series). An Aminex HPX-87H (300 mm×7.8 mm) column (Bio-Rad) was used with a mobile phase consisting of 0.013 NH₂SO₄ with a flow rate of 1.4 ml/min. Analyte peaks were detected and quantified using a refractive index detector (Waters 2414), identification of peaks was determined by reference to organic acid standard solutions purchased from Sigma. Table 1 below shows the fermentation evaluation of multiple A. succinogenes transformants, grown on sorbitol.

TABLE 1

| | | Fermentation Performance | | | |
|---|---|---|---|---|---|
| | | | | Yield (g | |
| | | | | succinic | |
| Strain | Con- | | Productivity | acid/g | |
| designation | struct | Titer (g/L) | (g/L-h | sugar) | n = |
| FZ53 | No | 109.23 ± 2.50 | 2.83 ± 0.12 | 0.99 ± 0.02 | 3 |
| | vector | | | | |
| FZ53 PLS88 | Empty | 107.00 ± 2.01 | 2.77 ± 0.06 | 0.97 ± 0.01 | 3 |
| | vector | | | | |
| FZ53 | zwf | 113.08 0.85 | 2.70 ± 0.17 | 1.03 ± 0.01 | 3 |
| p830.60 | | | | | |
| FZ53 | mdh | 120.53 ± 0.33 | 2.20 ± 0.14 | 1.10 ± 0.01 | 2 |
| pPISTONS1 | | | | | |
| FZ53 | zwf; | 120.72 ± 1.98 | 2.50 ± 0.11 | 1.12 ± 0.02 | 6 |
| p856.78 | mdh | | | | |

The transformation of A. succinogenes with an “empty” vector (pLS88) that contained no genes to be overexpressed by the host did not significantly impact the performance of the succinic acid fermentation when sorbitol was the carbon source. Over expression of either the zwf gene or the mdh gene as single genes (vectors p830.60 and pPISTONS1 respectively) increased the succinic acid yield. The highest yield was obtained with the overexpression of Mdh enzyme, however this high yield came at the expense of the productivity, which was lower in this transformant than in the Zwf expressing strain, the untransformed parent and the transformant with the empty vector.

The co-expression of zwf and mdh genes also lead to a high yield (equivalent to that observed for the mdh transformant), but also supported an increased productivity (not significantly different from the zwf transformant.

It is demonstrated that the co-expression of Zwf and Mdh enzymes promotes the formation of succinic acid with high productivity, yield and titer when sorbitol is the carbon source.

### Example 3

### Succinic Acid Production from Glucose by Overexpression of Zwf and Mdh Enzymes

Actinobacillus succinogenes strains (FZ53; FZ53/pLS88; FZ53/p830.60; FZ53/pISTONS1 and FZ53/p856.78) described in Example 1 were cultivated in 50 mL anaerobic vials in the same medium as described in Example 2 with the modification that the bottles were preloaded with 6 g MgCO₃ to maintain culture pH. The vials were inoculated with 2.5 mL (5% v/v inoculum) from an identical seed vial. Vials were incubated at 38° C. with constant shaking at 150 rev/min.

Analysis of culture filtrates was carried out as described in Example 2. An example of the analysis results are as follows (calculated at 46 hours): FZ53 pLS88 achieved a titer of 80.0 g/L, a productivity of 1.74 g/L-h, and yield of 0.76 g succinic acid/g sugar; FZ53 p830.60 (Zwf) achieved a titer of 100.6 g/L, a productivity of 2.19 g/L-h, and yield of 0.89 g succinic acid/g sugar; FZ53 pPISTONS1 (Mdh) achieved a titer of 80.2 g/L, a productivity of 1.87 g/L-h, and yield of 0.77 g succinic acid/g sugar; and FZ53 p878.56 (Zwf+Mdh) achieved a titer of 110.0 g/L, a productivity of 2.16 g/L-h, and yield of 0.96 g succinic acid/g sugar. See also Table 2, which summarizes the results of fermentation evaluation of A. succinogenes transformants on glucose.

TABLE 2

| Strain | | Improvements [%] | | |
|---|---|---|---|---|
| n = 3 | construct | titer | Productivity | yield |
| FZ53/pLS88 | empty | 100.0 | 100.0 | 100.0 |
| | vector | | | |
| FZ53/p830.60 | Zwf | 110.7 | 111.2 | 107.9 |
| FZ53/pISTONS1 | Mdh | 102.6 | 102.8 | 100.0 |
| FZ53/p856.78 | Zwf + Mdh | 117.5 | 114.0 | 122.4 |

Whilst the over expression of Zwf was confirmed to be beneficial, increasing both productivity and yield of succinic acid from glucose, the over expression of Mdh alone did not exhibit any beneficial impact. The co-expression of Zwf and Mdh did provide a benefit over the expression of Zwf alone, increasing the yield whilst not significantly decreasing the succinic acid productivity. Additional observations are provided below.

Succinic acid production using A. succinogenes is an anaerobic production process. The organism is a natural succinic acid producer. Production is stimulated by supplementation of culture with either H2 or more reduced substrates (e.g., sorbitol or mannitol), suggesting that reducing power (NADPH) limited succinic acid production in A. succinogenes. With this in mind, Zwf (an enzyme that directly generates NADPH and diverts glucose into the pentose phosphate pathway or Entner-Doudoroff pathway) was overexpressed in A. succinogenes. This intervention increased succinic acid fermentation performance in terms of yield. Therefore, the diversion of glucose from the glycolytic pathway through the pentose phosphate pathway or Entner-Doudoroff pathway (and increasing the intracellular NADPH pool) benefited succinic acid production.

Biochemical studies of A. succinogenes and comparison with succinic acid producing E. coli demonstrated that the enzymes involved in the conversion of phosphoenol pyruvate to succinic acid (phosphoenol pyruvate decarboxylase, malate dehydrogenase, fumarase, and fumarate reductase) are highly expressed in A. succinogenes, suggesting a mechanism behind the prodigious ability of A. succinogenes to accumulate succinic acid. In particular, malate dehydrogenase activity was found to be very high in A. succinogenes—greater than 10 fold higher than in E. coli (2100 nmol/min/mg protein c.f. 160 nmol/min/mg protein (Van der Werf, Arch. Microbiol., 167, 332-342 (1997)). Thus, malate dehydrogenase activity was not expected to limit the production of succinic acid in A. succinogenes. Indeed, Mdh over-expression alone failed to significantly improve titer, productivity, or yield compared to a control microorganism lacking a heterologous Mdh encoding polynucleotide. See Table 2. This observation appeared to confirm the conclusions drawn after the biochemical study of A. succinogenes, that Mdh was not a rate limiting step in the production of succinic acid in A. succinogenes as a result of its naturally high endogenous activity.

On the basis of the biochemical survey and the data reported in Table 2 for Mdh overexpression alone, there would be no expectation of a benefit of the co-expression of a polynucleotide encoding glucose-6-phosphate dehydrogenase (zwf) and a polynucleotide encoding a malate dehydrogenase (mdh) in A. succinogenes.

However, during characterization of a Zwf overexpressing strain of A. succinogenes, a peak in the HPLC by-product profile was surprisingly observed that was associated with, and increased with, Zwf overexpression. The peak was determined to correspond to oxaloacetate, a very labile compound that easily decarboxylates, dimerizes, and converts to malate. This suggested that, unexpectedly, when Zwf was overexpressed in A. succinogenes, Mdh became a limiting enzyme in succinic acid production, resulting in an accumulation of its substrate (oxaloacetate). Without wishing to be bound by a particular theory, the build-up of oxaloacetate as a byproduct diverts carbon flow, decreasing the succinic acid yield of the fermentation. Thus, contrary to biochemical in vitro studies that demonstrated that Mdh activity is high in A. succinogenes, enzyme activity in vivo was unexpectedly limited. Overproduction of Mdh in combination with Zwf overcame the limitation, avoiding the accumulation of oxaloacetate and increasing the yield of succinic acid.

This example demonstrates that the co-expression of Zwf and Mdh provides a benefit over the expression of either single enzyme when glucose is used as a carbon source. Mdh was previously shown not to be a limiting activity in the production of succinic acid by A. succinogenes. It was unexpectedly discovered that over-expression of Mdh lead to an improved performance in succinic acid production by A. succinogenes when Zwf was also overexpressed.

Some additional non-limiting embodiments are provided below to further exemplify the present invention.

In one embodiment the recombinant microorganism expresses glucose-6-phosphate dehydrogenase and malate dehydrogenase enzymes. In some embodiments a single enzyme or both enzymes are heterologous to the organism. In various embodiments, expression of the heterologous polynucleotide or overexpression of the polynucleotide encoding the glucose-6-phosphate dehydrogenase and/or malate dehydrogenase results in a greater than two-fold, five-fold, ten-fold, 25-fold, 50-fold, 100-fold, or 250-fold, or 500-fold increase in enzyme activity as compared with enzyme activity observed in a parent (unmodified, matched) microorganism.

The inventive microorganism in various aspects produces more organic acid (or produces organic acid more quickly or more efficiently) than an otherwise similar microorganism that does not comprise the heterologous polynucleotide(s) or does not overexpress the polynucleotide(s). In various embodiments, the microorganism produces at least 2%, at least 3%, at least 5%, at least 6%, at least 7%, at least 10%, at least 12%, at least 15%, at least 17%, at least 20%, at least 25%, or at least 30% more organic acid than an otherwise similar microorganism that does not comprise the heterologous polynucleotide(s) or does not overexpress the polynucleotide(s) in the same time frame. Alternatively or in addition, the microorganism demonstrates a level of productivity that is at least 2%, at least 3%, at least 5%, at least 6%, at least 7%, at least 10%, at least 12%, at least 15%, at least 17%, at least 20%, at least 25%, or at least 30% better than an unmodified (parent) microorganism. Alternatively or in addition, the microorganism demonstrates an increase in yield of at least 2%, at least 3%, at least 5%, at least 6%, at least 7%, at least 10%, at least 12%, at least 15%, at least 17%, at least 20%, at least 25%, or at least 30% compared to an unmodified (parent) microorganism.

In other embodiments both enzymes are expressed from A. succinogenes. In still other embodiments both enzymes are expressed from E. coli. In still other embodiments glucose-6-phoshphate dehydrogenase is encoded from E. coli and malate dehydrogenase is encoded by A. succinogenes. The gene combinations may be reversed. In other embodiments, the microorganisms expressing Zwf and Mdh include members of the Pasteurellaceae family. In still other embodiments, the microorganisms include Actinobacillus succinogenes, Bisgaard Taxon 6 and Bisgaard Taxon 10 or microorganisms that have more than 90% rRNA sequence identity to Actinobacillus succinogenes. The host cell expressing these genes may be A. succinogenes. In still other embodiments the genes may be expressed in separate organisms but both organisms included in the fermentation medium to produce organic acids. In one embodiment organic acids are succinic acid or propionic acid.

In some embodiments crude polyols may be used as the carbon source. In other embodiments, the fermentation broth or medium to grow the microorganisms include corn steep liquor, betaine, sodium, magnesium ions and combinations thereof.

The disclosure of the expression of enzymes that are used in the pentose phosphate pathway (or Entner-Doudoroff pathway) and the reductive tri-carboxylic acid cycle results in increased organic acid production than a single enzyme alone.

This application is intended to cover any adaptations or variations of the present subject matter. For example, although described primarily as a single recombinant organism that coexpresses zwf and mdh it can be envisaged that multiple organisms that each express zwf and mdh may be included in a single fermentation process to result in organic acid production. The invention includes variants or progeny of the microorganisms described herein that retain the phenotypic characteristics of the recombinant microbe. A substantially pure monoculture of the microorganism described herein (i.e., a culture comprising at least 80% or at least 90% of a desired microorganism) also is provided

What is claimed is:

1. A recombinant microorganism expressing glucose-6-phosphate dehydrogenase and malate dehydrogenase enzymes.

2. The recombinant microorganism of claim 1, wherein the recombinant organism is a succinic acid producing microorganism.

3. The recombinant microorganism of claim 1, comprising a 16S ribosomal RNA sequence with at least 90% identity to the 16S ribosomal RNA sequence of Actinobacillus succinogenes.

4. The recombinant microorganism of claim 1, which is Actinobacillus succinogenes, Bisgaard Taxon 6 or Bisgaard Taxon 10.

5. The recombinant microorganism of claim 1, comprising a heterologous or overexpressed polynucleotide encoding a glucose-6-phosphate dehydrogenase enzyme and a heterologous or overexpressed polynucleotide encoding a malate dehydrogenase enzyme.

6. The recombinant microorganism of claim 1, wherein the glucose-6-phosphate dehydrogenase or malate dehydrogenase are A. succinogenes enzymes.

7. The recombinant microorganism of claim 1, wherein the glucose-6-phosphate dehydrogenase and malate dehydrogenase is an A. succinogenes enzyme.

8. The recombinant microorganism of claim 1, wherein the glucose-6-phosphate dehydrogenase or malate dehydrogenase are E. coli enzymes.

9. The recombinant microorganism of claim 1, wherein the glucose-6-phosphate dehydrogenase and malate dehydrogenase is an E. coli enzyme.

10. The recombinant microorganism of claim 1, wherein the glucose-6-phosphate dehydrogenase comprises an amino acid sequence having at least about 40% sequence identity to the amino acid sequence set forth in SEQ ID NO: 2.

11. The recombinant microorganism of claim 1, wherein the malate dehydrogenase comprises an amino acid sequence having at least about 60% sequence identity to the amino acid sequence set forth in SEQ ID NO: 4.

12. The recombinant microorganism of claim 1, wherein the microorganism produces an increased amount of organic acid compared to a microorganism expressing glucose-6-phosphate dehydrogenase or malate dehydrogenase enzymes alone.

13. The recombinant microorganism of claim 1, wherein the microorganism is capable of producing succinic acid at concentrations of about 50 g/L to 150 g/L.

14. A process for organic acid production, which process comprises culturing the recombinant microorganism of claim 1 in a fermentation medium.

15. A method of producing organic acid comprising the step of:
culturing a recombinant microorganism with a carbon source under conditions favoring organic acid production, wherein the recombinant microorganism expresses at least one glucose-6-phosphate dehydrogenase and malate dehydrogenase enzymes.

16. The method of claim 15, wherein the recombinant microorganism expresses both glucose-6-phosphate dehydrogenase and malate dehydrogenase enzymes.

17. A method of producing organic acid comprising culturing the recombinant microorganism of claim 1 with a carbon source under conditions favoring organic acid production.

18. The method of claim 16, wherein the organic acid is succinic acid or propionic acid.

19. The method of claim 18, wherein the organic acid is succinic acid.

20. The method of claim 16, wherein the carbon source is glucose or sorbitol.

21. The method of claim 17, wherein the organic acid is succinic acid or propionic acid.

22. The method of claim 21, wherein the organic acid is succinic acid.

23. The method of claim 17, wherein the carbon source is glucose or sorbitol.
